(19) United States
(12) Patent Application Publication
(10) Pub. No.: US 2010/0009445 A1
(43) Pub. Date: Jan. 14, 2010

(54) RARE EARTH NANOPARTICLES

(75) Inventors: Chittaranjan Patra, Rochester, MN (US); Debabrata Mukhopadhyay, Rochester, MN (US); Resham Bhattacharya, Rochester, MN (US); Priyabrata Mukherjee, Rochester, MN (US); Nicholas E. Vlahakis, Rochester, MN (US)

Correspondence Address: SHEPPARD, MULLIN, RICHTER & HAMPTON LLP, 990 Marsh Road, Menlo Park, CA, 94025, US

(73) Assignee: Mayo Foundation for Medical Education and Research, Rochester, MN (US)

(21) Appl. No.: 12/376,272

(22) PCT Filed: Aug. 14, 2007
(86) PCT No.: PCT/US2007/075926

§ 371 Date: Jun. 22, 2009

Related U.S. Application Data
(60) Provisional application No. 60/837,807, filed on Aug. 14, 2006.

(51) Int. Cl. C12N 5/02; A62D 3/10; C01F 17/00

(52) U.S. Cl. 435/375; 204/157.43; 423/263; 977/773

(57) ABSTRACT

This document provides methods and materials related to rare earth particles such as rare earth nanorods (e.g., inorganic lanthanide hydroxide nanorods). For example, rare earth (e.g., lanthanide) particles such as europium hydroxide nanorods, methods and materials for making rare earth particles (e.g., europium hydroxide nanorods), and methods and materials for using rare earth particles (e.g., europium hydroxide nanorods) as an imaging agent and/or to promote angiogenesis are provided.

### CROSS-REFERENCE TO RELATED APPLICATIONS

This Application claims priority to U.S. Provisional Application No. 60/837,807, filed Aug. 14, 2006.

### STATEMENT AS TO FEDERALLY SPONSORED RESEARCH

This invention was made with government support under grant numbers CA78383 and HL70567 awarded by National Institutes of Health. The government has certain rights in the invention.

### BACKGROUND

1. Technical Field

This document relates to methods and materials involved in nanoparticles (e.g., rare earth nanorods). For example, this document relates to materials and methods involved in neodymium, samarium, europium, gadolinium, and terbium nanoparticles.

2. Background Information

Nanotechnology is a rapidly expanding into biomedical research. Nanobiotechnology is opening new avenues in bioimaging, medical diagnostics, and disease therapy. Bio-imaging with inorganic fluorescent nanoparticle probes recently attracted widespread interest in biology and medicine.

### SUMMARY

This document provides methods and materials related to rare earth particles such as rare earth nanorods (e.g., inorganic lanthanide hydroxide nanorods). For example, this document provides neodymium hydroxide [Nd^{III}(OH)₃], samarium hydroxide [Sm^{III}(OH)₃], europium hydroxide [Eu^{III}(OH)₃], gadolinium hydroxide [Gd^{III}(OH)₃], and terbium hydroxide [Tb^{III}(OH)₃] nanorods. These nanorods can be prepared using a microwave technique that is simple, fast, clean, efficient, economical, non-toxic, and eco-friendly. The europium hydroxide nanorods provided herein can be fluorescent, can enter cells, and can retain their fluorescent properties once they have entered cells. In addition, the europium hydroxide nanorods provided herein can be used to visualize the internalization of drugs or biomolecules attached to the nanorods into cells for imaging, therapeutic, and/or diagnostic purposes. The europium hydroxide nanorods provided herein can be non-toxic, fluorescent, inorganic, Europium(III) hydroxide nanorods and can be used as pro-angiogenic agents in vivo.

The process of angiogenesis can play a role in embryogenesis, wound healing, and tumor genesis through the growth of new blood vessels from pre-existing vasculature. The europium hydroxide nanorods provided herein can be used to promote angiogenesis in tissues such as ischemic tissues. In some cases, europium hydroxide inorganic fluorescent nanorods can be used as a pro-angiogenic agent instead of or in combination with vascular endothelial growth factor (VEGF) and basic fibroblast growth factor (BFGF). The europium hydroxide nanorods provided herein can be non-toxic nanorods as observed by a cell proliferation assay, a cell cycle assay, and/or a CAM assay and can induce endothelial cell proliferation. The europium hydroxide nanorods provided herein can be used to treat heart or limb ischemic tissues in humans. Like Eu^{III}(OH)₃ nanorods, Nd^{III}(OH)₃, Sm^{III}(OH)₃, and Tb^{III}(OH)₃ nanorods are non-toxic, as observed by a cell proliferation assay.

In comparison to organic dyes (Fluorescein, Texas Red™, Lissamine Rhodamine B, Tetramethylrhodamine, etc.) and fluorescent proteins (Green fluorescent protein, GFP), the inorganic fluorescent europium hydroxide nanorods provided herein can have several unique optical and electronic properties such as size- and composition-tunable emission from visible to infrared wavelengths, a large stokes shift, a symmetric emission spectrum, simultaneous excitation of multiple fluorescent colors, very high levels of brightness and photostability.

In general, one aspect of this document features a method for making europium hydroxide nanorods. The method comprises, or consists essentially of, microwave heating a mixture of Ln^{III}(NO₃)₃ (where Ln=Nd, Sm, Eu, Gd, or Tb) and aqueous ammonium hydroxide. The lanthanide hydroxide [Ln^{III}(OH)₃] nanorods can be between 10 and 500 nm in length. The diameter of the lanthanide hydroxide nanorods can be between 1 and 100 nm.

In another aspect, this document features lanthanide hydroxide nanorods having a length between 10 and 500 nm and a diameter between 1 and 100 nm, wherein the nanorods promote angiogenesis.

In another aspect, this document features a method of promoting angiogenesis, wherein the method comprises, or consists essentially of, contacting cells with europium hydroxide nanorods. The europium hydroxide nanorods can be between 10 and 500 nm in length. The diameter of the europium hydroxide nanorods can be between 1 and 100 nm.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph plotting XRD patterns of microwave assisted, as-synthesized europium hydroxide [Eu^{III}(OH)₃] at different reaction times: (a) 5 minutes, (b) 10 minutes, (c) 20 minutes, (d) 40 minutes, and (e) 60 minutes.

FIG. 2 is a graph plotting XRD patterns of microwave assisted, as-synthesized neodymium hydroxide [Nd^{III}(OH)₃], samarium hydroxide [Sm^{III}(OH)₃], gadolinium hydroxide [Gd^{III}(OH)₃], and terbium hydroxide [Tb^{III}(OH)₃] after microwave heating for 60 minutes.

FIG. 3 contains graphs of thermogravimetric analyses (TGA; panels A and C) and differential scanning calorimetric (DSC) plots (panels B and D) of microwave-assisted, as-synthesized europium hydroxide nanorods. Panels A and B: 5 minute sample. Panels C and D: 60 minute sample.

FIG. 4 contains graphs of thermogravimetric analyses (TGA) of microwave assisted, as-synthesized neodymium hydroxide [Nd^{III}(OH)₃] (panel A), samarium hydroxide [Sm^{III}(OH)₃] (panel B), gadolinium hydroxide [Gd^{III}(OH)₃] (panel C), and terbium hydroxide [Tb^{III}(OH)₃] (panel D) after microwave heating for 60 minutes.

FIG. 5 contains TEM images of as-synthesized Eu^{III}(OH)₃ nanorods at various times of microwave heating: 5 minutes (panel A), 10 minutes panel B), 20 minutes (panel C), 40 minutes (panel D), 60 minutes (panel E; at low magnification), and 60 minutes (panel F; at higher magnification).

FIG. 6 contains TEM images of as-synthesized neodymium hydroxide [Nd^{III}(OH)₃] nanorods after microwave heating for the following lengths of time: 1 minute (panel A), 5 minutes (panel B), 10 minutes (panel C), 20 minutes (panel D), 40 minutes (panel E), and 60 minutes (panel F).

FIG. 7 contains TEM images of as-synthesized samarium hydroxide [Sm^{III}(OH)₃] nanorods after microwave heating for the following lengths of time: 1 minute (panel A), 5 minutes (panel B), 10 minutes (panel C), 20 minutes (panel D), 40 minutes (panel E), and 60 minutes (panel F).

FIG. 8 contains TEM images of as-synthesized Gd^{III}(OH)₃ (panel A) and Tb^{III}(OH)₃ (panel B) nanoparticles, some of which can be nanorods, after microwave heating for 60 minutes.

FIG. 9, panels A and B, contain two graphs of an excitation spectra of microwave assisted, as-synthesized Eu(OH)₃. The graph in panel B is a higher magnification of the graph in panel A. FIG. 9C contains a graph of an emission spectrum of microwave assisted, as-synthesized Eu^{III}(OH)₃.

FIG. 10 is a graph plotting the emission spectra of Eu^{III}(OH)₃ nanorods inside endothelial cells treated with the following concentrations of nanorods: a=5 μg/mL, b=10 μg/mL, c=20 μg/mL, d=50 μg/mL, and e=100 μg/mL). There was no emission peak for the control experiment.

FIG. 11 contains DIC microscopy pictures of HUVEC with nanorods and without nanorods. Panel A: control HUVEC with no treatment; no nanorods are observed. Panels B-D: HUVEC treated with Eu(OH)₃ nanorods at the following concentrations. Panel B: 20 μg/mL, panel C: 50 μg/mL, and panel D: 100 μg/mL. Nanorods inside the cells are marked by white arrows (panels B-D) in a few places.

FIG. 12 contains fluorescence (left panels) and corresponding phase images (right panels) of endothelial cells (HUVEC). Panel A contains images of control endothelial cells with no treatment. The slight green color is due to auto fluorescence in panel A. Panels B and C contain confocal microscopy images of endothelial cells treated with Eu(OH)₃ nanorods at 20 μg/mL (panel B) or 50 μg/mL (panel C). The arrows indicate the fluorescence of particles located inside the cells.

FIG. 13 contains TEM photographs of Eu^{III}(OH)₃ nanorods inside the cytoplasmic compartments of endothelial cells. The images of the nanorods were visualized by TEM inside the cytoplasmic compartments of HUVECs treated with 20 μg/mL (panels A-C) or 50 μg/ml (panels D-F) of nanorods. Panels B and C contain enlarged images from panel A. Panels E and F contain enlarged images from panel D.

FIG. 14 contains TEM photographs of Tb^{III}(OH)₃ nanorods inside the cytoplasmic compartments of endothelial cells. The images of the nanorods were visualized by TEM inside the cytoplasmic compartments of HUVECs treated with 50 μg/mL of nanorods. Panels B, C, and D contain enlarged images from panel A.

FIG. 15 contains a graph plotting the effect of different concentrations of europium hydroxide nanorods on serum-starved HUVEC, observed using a [³H] Thymidine incorporation assay, and represented as fold stimulation. Eu-20, -50, -100 indicate cells treated with 20, 50, and 100 μg/mL of Europium hydroxide, respectively. VF indicates cells treated with VEGF (10 ng/mL). The data represent fold stimulation and are presented as the mean±SD of three separate experiments performed in triplicate. The data are statistically significant where p≦0.05.

FIG. 16 contains a graph plotting the effect of different concentrations of Neodymium hydroxide nanorods on serum-starved HUVEC, observed using a [³H] Thymidine incorporation assay and represented as fold stimulation. Nd-10, -20, -50 indicate cells treated with 10, 20, and 50 μg/mL of neodymium hydroxide, respectively.

FIG. 17 contains a graph plotting the effect of different concentrations of samarium hydroxide nanorods on serum-starved HUVEC, observed by a [³H] Thymidine incorporation assay and represented as fold stimulation. Sm-20, -50, -100 indicate cells treated with 20, 50, and 100 μg/mL of samarium hydroxide, respectively.

FIG. 18 contains a graph plotting the effect of different concentrations of terbium hydroxide nanorods on serum-starved HUVEC, observed by a [³H] Thymidine incorporation assay and represented as fold stimulation. Tb-20, -50, -100 indicate cells treated with 20, 50, and 100 μg/mL of terbium hydroxide, respectively. TbN indicates cells treated with terbium nitrate at a concentration of 20 μg/mL.

FIG. 19 contains photographs of HUVECs subjected to a Tunnel assay for apoptosis. Panels A-C contain images of HUVECs treated with camptothecin for four hours at 37° C. to induce apoptosis. The camptothecin treated cells served as a positive control. TMR red-stained nuclei of HUVECs appear red in color due to presence of apoptotic cells (panel A). The DAPI-stained nuclei appeared blue (panel B). Panel C contains a merged picture of panels A and B. Panels D-F contain images of untreated HUVECs. Panels G-T contain images of HUVECs treated with Eu^{III}(OH)₃ at 50 μg/mL for 20 hours of incubation at 37° C. Panels J-L contain images of HUVECS treated with Eu^{III}(OH)₃ at 100 μg/mL for 20 hours of incubation at 37° C. The nuclei of the HUVECs shown in the first column (panels A, D, G, and J) were stained with TMR red. Red staining was not observed due to the absence of apoptotic cells. The DAPI-stained nucleic of the cells shown in the second column (panels B, F, H, and K) appear blue. Each row of the third column (panels C, F, I, and L) contains a merged image of the two images in the first and second columns of the same row.

FIG. 20 is a graph of a cell cycle analysis of endothelial cells (HUVEC) in the presence of different doses of Eu^{III}(OH)₃ nanorods (0-100 μg/ml). S-phase (S) is highest at a concentration of 50 μg/mL of Eu^{III}(OH)₃ nanorods. The data are presented as the mean±SD of 3 separate experiments performed in triplicate and are statistically significant where p≦0.05.

FIG. 21 contains a Western blot analyzing phospho-map kinase and total map kinase. Lysates were prepared from HUVECs that were treated with Eu^{III}(OH)₃ nanorods (50 μg/mL) for the indicated times (5 minutes to 6 hours), or that were treated with VEGF (10 ng/mL) for 5 minutes (panel A). Panel B contains a Western blot analyzing phospho-map kinase and total map kinase in HUVECs that were mock-treated (control) or treated with Eu(OH)₃ nanorods at 20 μg/mL (E20) or 50 μg/mL (E50) for 24 hours.

FIG. 22 contains images of HUVECs analyzed for reactive oxygen species (ROS). Panels A-C contain images of control untreated HUVECs. Panels D-F contain images of HUVECs treated with 100 μM/mL of tert-butyl hydroperoxide (TBHP) as a positive ROS inducer. Panels G-L contain images of HUVECs treated with 20 μg/mL (panels G-T) or 50 μg/mL (panels J-L) of Eu^{III}(OH)₃ nanorods.

FIG. 23 contains photographs of chicken chorioallantoic membranes (CAMs) treated with TE (Tris-EDTA) buffer (panel A), VEGF (50 ng; panel B), or 1 μg or 10 μg of nanorods in TE buffer (panels C and D). Panel E contains a graph plotting CAM assay results. Angiogenesis was quantified by counting branch points arising from tertiary vessels from a minimum of 10 specimens from three separate experiments.

### DETAILED DESCRIPTION

This document provides methods and materials related to rare earth particles such as rare earth nanorods. For example, this document provides rare earth (e.g., lanthanide) particles such as neodymium (Nd), samarium (Sm), europium (Eu), gadolinium (Gd), and terbium (Tb) hydroxide nanorods, methods and materials for making rare earth particles (e.g., neodymium, samarium, europium, gadolinium, and terbium hydroxide nanorods), and methods and materials for using rare earth particles (e.g., neodymium, samarium, europium, gadolinium, and terbium hydroxide nanorods) as an imaging agent and/or to promote angiogenesis.

Lanthanide (e.g., neodymium, samarium, europium, gadolinium, and terbium) hydroxide nanoparticles (e.g., nanorods) provided herein can have any dimensions. For example, the europium hydroxide nanorods provided herein can have a length between 50 nm and 500 nm (e.g., between 100 nm and 400 nm, between 150 nm and 350 nm, and between 200 nm and 300 nm), and can have a thickness between 10 nm and 100 nm (e.g., between 20 nm and 90 nm, between 25 nm and 75 nm, or between 30 nm and 50 nm). Any appropriate method can be used to make lanthanide hydroxide nanoparticles. For example, a microwave technique such as that described herein can be used to make lanthanide (e.g., neodymium, samarium, europium, gadolinium, and terbium) hydroxide nanorods.

In some cases, the lanthanide hydroxide nanoparticles provided herein can be combined with drugs or other therapeutic agents for delivery to a mammal (e.g., a human). For example, a drug can be covalently linked to an europium hydroxide nanoparticle (e.g., nanorod). In such cases, the europium hydroxide nanoparticles (e.g., nanorods) can be used to track the location and/or concentration of the drug within a mammal. Examples of therapeutic agents that can be combined with lanthanide hydroxide nanoparticles include, without limitation, polypeptides, antibodies, C225, gemcitabine, cisplatin, and organic drug molecules containing an active functional group.

Any appropriate method can be used to combine lanthanide hydroxide nanoparticles with therapeutic agents. For example, a therapeutic agent can be conjugated to a lanthanide hydroxide nanoparticle. Before conjugating a therapeutic agent (e.g., a drug molecule) with a lanthanide hydroxide nanoparticle (e.g., a europium hydroxide nanorod), the surface of the nanoparticle (e.g., nanorod) can be modified with an active functional group (e.g., an amino or mercapto group). For example, aminopropyl trimethoxy silane (APTMS) or mercapto-propyl trimethoxy silane (MPTMS) can be used to functionalize the surface of lanthanide hydroxide nanorods, as described elsewhere (Feng et al., Anal. Chem., 75:5282-5286 (2003)). In some cases, nanoparticles (e.g., nanorods) can be functionalized using a microwave technique such as that described herein. Surface modified lanthanide hydroxide nanoparticles can be combined with different therapeutic agents (e.g., organic drug molecules, polypeptides, or antibodies) by covalent bond formation.

As described herein, lanthanide hydroxide nanoparticles such as europium hydroxide nanorods can be used to promote angiogenesis within a mammal. For example, a mammal can be identified as needing a pro-angiogenic agent. Once identified, lanthanide hydroxide nanoparticles provided herein can be administered to the mammal. Such an administration can be a systemic or local administration. For example, europium hydroxide nanorods can be directly injected into tissue in need of angiogenesis. Following administration, the mammal can be monitored to determine whether or not angiogenesis was promoted or to determine whether or not additional administrations are needed.

The invention will be further described in the following examples, which do not limit the scope of the invention described in the claims.

### EXAMPLES

### Example 1

### Materials

Neodium (III) nitrate hexahydrate (99.9%), Samarium (III) nitrate hexahydrate (99.99%), Europium (III) nitrate hydrate [Eu(NO₃)₃.xH₂O, 99.99%], Gadolinium (III) nitrate hexahydrate (99.999%), Terbium (III) nitrate hexahydrate (99.999%), and aqueous ammonium hydroxide [aq.NH₄OH, 28-30%] were purchased from Aldrich (USA) and were used without further purification. [³H] Thymidine was purchased from Amersham Biosciences (Piscataway, N.J.). Phosphate Buffered Saline (PBS) without calcium and magnesium was purchased from Cellgro Mediatech, Inc. (Herndon, Va.). Endothelial Cell Basal Medium (EBM), without anti-microbial agents, Trypsin/EDTA (0.25 mg/mL), Trypsin Neutralizing Solution (TNS), and a set of 5% of fetal bovine serum (FBS), 0.4% of bovine brain extract, and 0.1% of gentamicin sulfate amphotericin-B, were obtained from Cambrex Bio Science Inc. (Walkersville, Md.) and used to make EBM complete media. Falcon tissue culture dishes were purchased from Beckon Dickinson Labware (Beckon Dickinson and Company, N.J., USA). An in situ cell death detection kit, TMR red, for use in a Tunnel assay was purchased from Roche (Cat. No. #12 156 792 910). Monoclonal mouse IgG (Cat. No # OP72-100UG), anti-phospho map kinase (rabbit polyclonal IgG, Cat. No. # 07-467) antibody, and anti-mouse IgG or anti-rabbit IgG-HRP (Cat. # Sc-2301) were purchased from Santa Cruz Biotechnology (Santa Cruz, Calif. USA). The Image-iT™ LIVE Green Reactive Oxygen Species (ROS) Detection Kit (I36007) was purchased from Invitrogen Molecular Probes (Eugene, Oreg.).

### Example 2

### Microwave-Assisted Synthesis of

Europium Hydroxide [Eu(OH)₃)] Nanorods

Lanthanide nitrate and aqueous ammonium hydroxide (28-30% A.C.S reagent) were purchased from Aldrich Co. and Sigma-Aldrich, respectively, and used as received without further purification. Ln^{III}(OH)₃, where Ln=Nd, Sm, Eu, Gd, or Tb, nanorods were prepared by microwave heating a mixture of an aqueous solution of Ln(III) nitrate and aq.NH₄OH at atmospheric pressure in an open reflux system. In a typical synthesis, 10 mL of aqueous NH₄OH was added to 20 mL 0.05 M of an aqueous solution of Ln(III) nitrate (pH=5.5) in a 100 mL round-bottomed flask. A colloidal precipitate, without any special morphology, was obtained upon the addition of NH₄OH to Ln(III) nitrate solution. The pH of the solution before and after the reaction was 9.4 and 7.5, respectively. The samples were irradiated for 1 to 60 minutes with 60% of the instrument's power (on/off irradiation cycles ratio of 3/2) in order to control the reaction and reduce the risk of superheating of the solvent. The microwave refluxing apparatus was a modified domestic microwave oven (GOLD STARR 1000 W, LA Electronics, Inc., Huntsville, Ala.) with a 2.45 GHz output power, as described elsewhere (Matsumura Inoue et al., Chem. Lett., 2443 (1994)). In the post-reaction treatment, the resulting products were collected, centrifuged at 15000 rpm, washed several times using distilled water, and then dried overnight under vacuum at room temperature. The yield of the as-prepared products was more than 95% for all of the lanthanide hydroxide nanoparticles. The above experiments were conducted several times and exhibited good reproducibility.

### Example 3

### Experimental Procedures

The following cell culture experiments were performed: differential interference contrast (DIC) microscopy, confocal microscopy, determination of reactive oxygen species (ROS), tunnel assay (apoptosis), fluorescence spectroscopy, transmission spectroscopy, and a trypan blue exclusion dye test.

Human umbilical vein endothelial (HUVEC) cells were cultured at 10⁵ cells/2 mL in six well plates for about 24 hours at 37° C. and 5% CO₂ in EBM complete media. For investigating the cellular localization, cells were plated on glass cover slips and grown up to 90% confluence in six well plates and then incubated with Eu^{III}(OH)₃ nanorods at a concentration range of 20-100 μg/mL. After 24 hours of incubation, the cover slips were rinsed extensively with phosphate buffer saline, and the cells were fixed with freshly prepared 4% para-formaldehyde in PBS for 15 minutes at room temperature and then re-hydrated with PBS. Once all cells were fixed, the cover slips with the cells were mounted onto glass slides with Fluor Save mounting media and examined using DIC and confocal microscopy. For investigating the formation of reactive oxygen species (ROS), the Image-iT™ LIVE Green Reactive Oxygen Species Detection Kit (Cat. No. #I36007; Molecular Probes, USA) was used according to the manufacturer's instructions with the treated and untreated cells finally mounted onto glass slides with Fluor Save mounting media and examined using confocal microscopy. For a tunnel assay, cells were mounted onto glass slides with mounting media with DAPI (4′-6-Diamidino-2-phenylindole) and examined using confocal microscopy according to the manufacturer's instructions (Roche, Cat. No. # 12 156 792 910).

In another set, HUVEC cells (10⁵ cells/2 mL) were cultured in six well plates and treated with Eu^{III}(OH)₃ or Tb^{III}(OH)₃ nanorods in EBM complete media without cover slips. After 24 hours of incubation with nanorods, the cells were washed with PBS, trypsinized, and neutralized. The cells were washed by centrifugation, re-suspended in the PBS, and examined using fluorescence spectroscopy and TEM. Cell viability for another set of cells was determined through staining with Trypan Blue, and cells were counted using a hemocytometer.

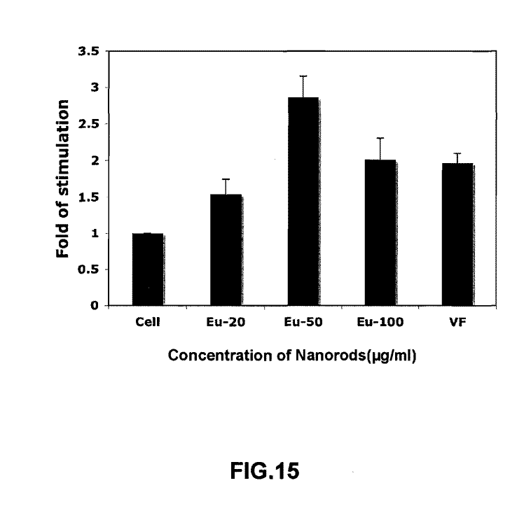

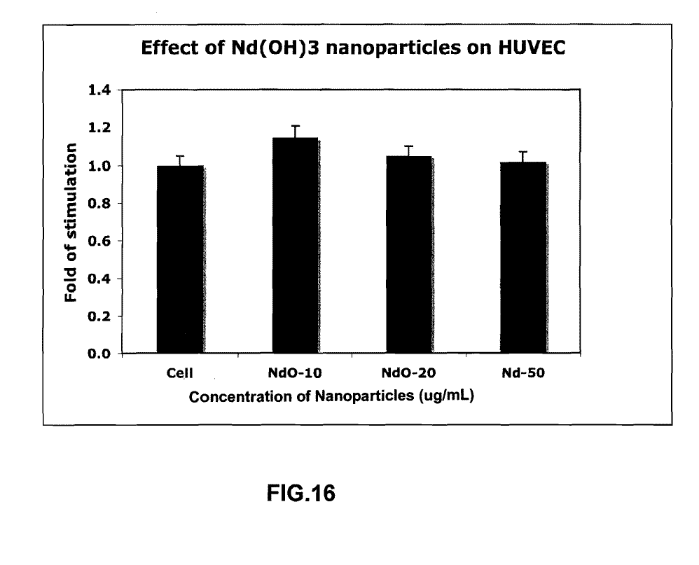

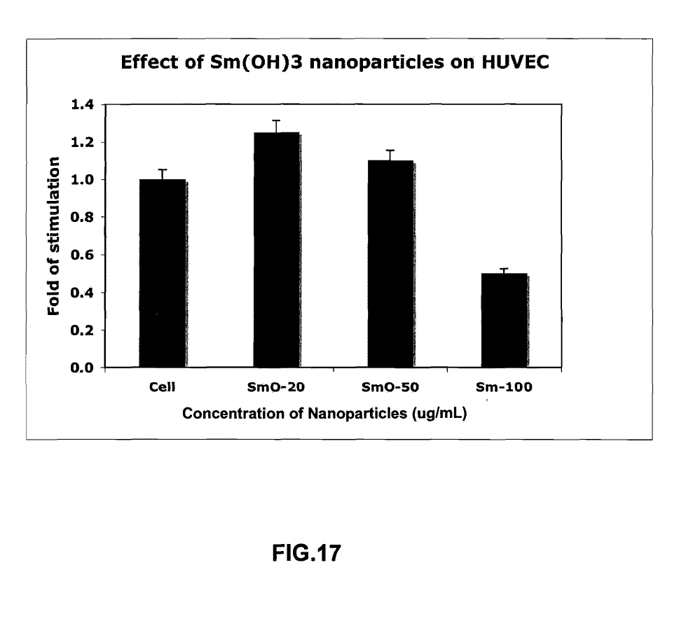

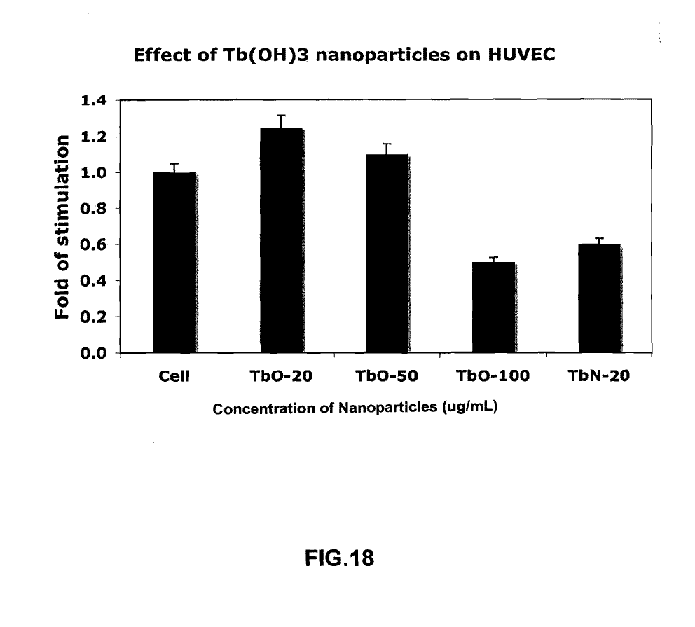

Cell viability and cell proliferation tests: An in vitro toxicity analysis in terms of inhibition of proliferation using [³H]thymidine incorporation assay to normal endothelial cells (HUVEC) was performed as described elsewhere (Basu et al., Nat Med., 7:569 (2001)). Briefly, endothelial cells (HUVEC; 2×10⁴) were seeded in 24-well plates, cultured for one day in EBM, serum-starved (0.1% serum) for 24 hours, and then treated with different concentrations (0, 20, 50 or 100 μg/mL) of Eu^{III}(OH)₃ (FIG. 15), Sm^{III}(OH)₃ (FIG. 17), or Tb^{III}(OH)₃ (FIG. 18). HUVECs also were treated with different concentrations (0, 10, 20 and 50 μg/mL) of Nd^{III}(OH)₃ (FIG. 16). After 24 hours, 1 μCi [³H] thymidine was added in each well. Four hours later, cells were washed with cold PBS, fixed with 100% cold methanol, and collected for the measurement of trichloroacetic acid-precipitable radioactivity. Experiments were repeated three times.

Apoptosis assay: To perform a tunnel apoptosis assay, cells were seeded into 6-well plates at a density of 10⁵ cells/2 mL of medium per well and grown overnight on cover slips. The cells were incubated with Eu^{III}(OH)₃ nanorods at different concentrations, mounted onto glass slides with Fluor Save mounting media with DAPI (4′-6-Diamidino-2-phenylindole), and examined using confocal microscopy according to the manufacture's instructions (Roche, USA, Cat. No. # 12 156 792 910). The red colored apoptotic cells were visualized using a microscope, counted (6 fields per sample), and photographed using digital fluorescence camera.

Cell cycle: The cell cycle analysis was performed according to the following standard procedure. DNA content was measured after staining cells with propidium iodide (PI). After treatment of Eu^{III}(OH)₃ nanorods, HUVEC cells were washed in PBS (3×) and fixed in 95% ethanol for 1 hour. Cells were re-hydrated, washed in PBS, and treated with RNaseA (1 mg/mL) followed by staining with PI (100 μg/mL). Similar experiments were done with control cells (No Eu^{III}(OH)₃ nanorods). Flow cytometric quantification of DNA was done by a FACScan (Becton-Dickinson), and the data analysis was carried out using Modfit software.

Western blot for Map kinase Phosphorylation: Harvested HUVEC cells were washed two times with cold PBS and lysed with ice-cold radioimmunoprecipitation (RIPA) buffer with freshly added 0.01% protease inhibitor cocktail (Sigma). After being incubated on ice for 10 minutes, the cells were centrifuged at 13,000 rpm for 10 minutes at 4° C. After measurement of protein concentration using a photometric method, 20 μg of protein were electrophoresed on a 10% (Tris-HCl) preparative polyacrylamide gel under reducing conditions and transferred to a nitrocellulose membrane by wet blotting. Membranes were cut into strips, blocked in 5% dry milk in tris-buffered saline for 1 hour, incubated overnight with monoclonal mouse IgG (Cat. No # OP72-100UG) for total map kinase or with anti-phospho map kinase (rabbit polyclonal IgG, Cat. No. # 07-467) antibodies, and then with HRP-coupled secondary antibodies (anti-mouse IgG or anti-rabbit IgG-HRP (Cat. #Sc-2301)) at 37° C. for 40 minutes. Detection was performed using a chemiluminescent substrate.

CAM assay: Chick eggs were maintained in a humidified 39° C. incubator (Lyon Electric, Calif.), as described elsewhere (Vlahakis et al., J. Biol. Chem., 282(20):15187-15196 (2007)). Pellets containing 0.5% methylcellulose plus recombinant human VEGF-A (50 ng) or bFGF (150 ng) were placed onto the CAM's of 10-day-old chick pathogen-free embryos (SPAFAS; Charles River Laboratories, Wilmington, Mass.). The CAM's were exposed by cutting a small window in the egg shell to facilitate application of the pellet. Relevant antibodies or agonist/antagonist compounds were applied to the site 24 hours after stimulation with VEGF polypeptides. In some cases, a suspension of europium hydroxide nanorods in Tris-EDTA buffer was applied using a micro-syringe. CAMs were imaged on day 13 either following fixation and excision or with real time live imaging using a digital camera (Canon Supershot6) attached to a Zeiss stereomicroscope. Angiogenesis was quantified by counting branch points arising from tertiary vessels from a minimum of ten specimens from three separate experiments.

### Example 4

### Characterization Techniques

The following techniques were performed to characterize Eu^{III}(OH)₃ nanorods.

X-ray diffraction (XRD): The structure and phase purity of the as-synthesized samples were determined by X-ray diffraction (XRD) analysis using a Bruker AXS D8 Advance Powder X-ray diffractometer (using CuK_{α}λ=1.5418 Å radiation).

Thermo-gravimetric (TG) and Differential Scanning Calorimetric (DSC)

Analysis: TGA of the as-synthesized sample was carried out under a stream of nitrogen at a heating rate of 10° C./minute from 30° C. to 700° C. using a METTLER TOLEDO TGA/STDA 851. DSC analysis of the as-synthesized sample was carried out on METTLER TOLEDO TC15 using a stream of nitrogen (20 mL/minute) at a heating rate of 4° C./minute in a crimped aluminum crucible from 30° C. to 600° C.

Transmission electron microscopy (TEM) study: The particle morphology (microstructures of the samples) was examined with TEM on a FEI Technai 12 operating at 80 KV. To visualize the internalization of particles inside the cytoplasmic compartment of cells using TEM, procedures described elsewhere were followed (McDowell and Trump, Arch. Path. Lab. Med., 100: 405 (1976) and Spurr, J. Ultrastruct. Res., 26:31 (1969)).

Fluorescence Spectroscopy The excitation and emission (fluorescence) spectra were recorded on Fluorolog-3 Spectrofluorometer (HORIBA JOBINYVON, Longjumeau, France) equipped with xenon lamp as the monochromator excitation source.

Differential interference contrast (DIV) microscopy: After fixation of cells on cover slips, the cells were mounted onto glass slides with Fluor Save mounting media and examined for DIC. Pictures were captured by AXIOCAM high-resolution digital camera using AXIOVER 135 TV microscope (ZEISS, Germany).

Confocal Fluorescence Microscopy for Eu^{III}(OH)₃, Tunel assay, and ROS: Two dimensional confocal fluorescence microscopy images were collected through use of LSM 510 confocal laser scan microscope (Carl Zeiss, Inc., Oberkochcn, Germany) with C-Apochromat 63×/NA 1.2 water-immersion lense, in conjunction with an Argon ion laser (488 nm excitation). The fluorescence emissions for Eu^{III}(OH)₃ nanorods, untreated cells, and cells treated with Eu^{III}(OH)₃ nanorods were collected through a 515 nm long pass filter.

For tunnel assay, after mounting the cells onto glass slides with DAPI, the images were collected through use of LSM 510 confocal laser scan microscope (Carl Zeiss, Inc., Oberkochcn, Germany) with C-Apochromat 63 X/1.2 na water-immersion lense. The fluorescence emissions were collected through a 385-470 nm band pass filter in conjunction with an Argon ion laser excitation of 364 nm for DAPI stained blue nuclei. The fluorescence emissions were collected through a 560-615 nm band pass filter in conjunction with HeNel ion laser excitation of 543 nm for TMR red stained apoptotic nuclei.

For ROS, the images were collected through use of LSM 510 confocal laser scan microscope (Carl Zeiss, Inc., Oberkochcn, Germany) with C-Apochromat 63×/1.2 na water-immersion lense. The green fluorescence (oxidation product of carboxy-H₂DCFDA) emissions were collected through a 505-550 nm band pass filter in conjunction with an Argon ion laser excitation of 488 nm. The blue fluorescence emissions for Hoechst 33342 stained blue nuclei were collected through a 385-470 nm band pass filter in conjunction with Argon ion laser excitation of 364 nm.

### Example 5

### Inorganic Fluorescent Nanorods and Their Pro-angiogenic Properties

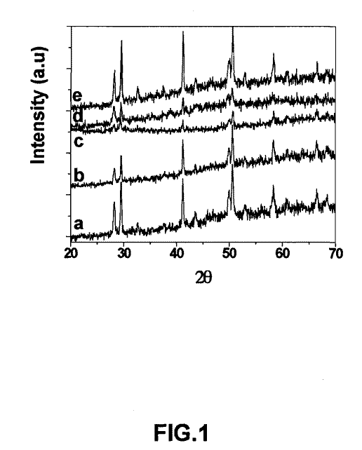

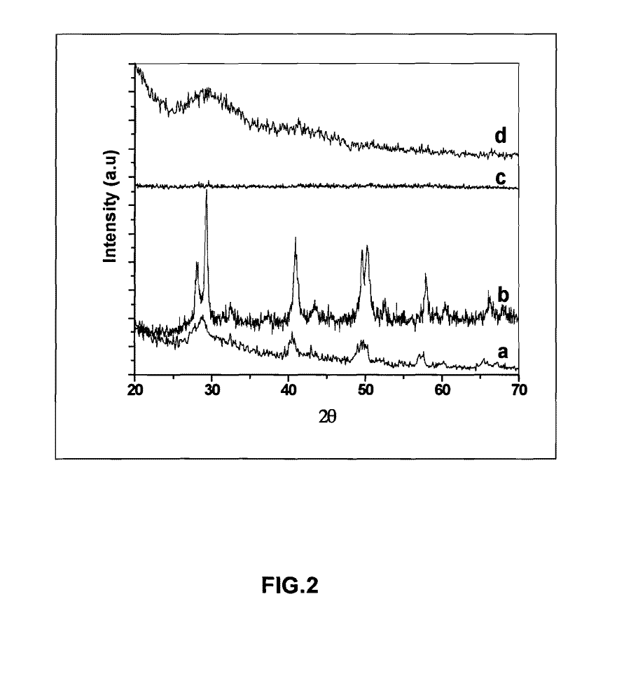

X-ray Diffraction Studies: The crystal structures of the as-synthesized materials were identified by X-ray diffraction (XRD) analysis (FIG. 1A-E). Curves a-, b-, c-, d-, and e- in FIG. 1 indicate the XRD patterns of the formation of as-synthesized europium hydroxide Eu^{III}(OH)₃, obtained after 5 minutes, 10 minutes, 20 minutes, 40 minutes, and 60 minutes of MW irradiation, respectively, by the interaction of europium(III) nitrate and ammonium hydroxide in water as solvent. The XRD patterns of as-synthesized materials, synthesized at different times, indicated that the products were crystalline. All reflections could be distinctly indexed to a pure hexagonal phase of Eu^{III}(OH)₃ materials. The diffraction peaks were consistent with the standard data files (the JCPDS card No. 01-083-2305) for all reflections. Similarly, the structures of microwave assisted, as-synthesized products (after 60 minutes of microwave irradiation) were analyzed by X-ray diffraction (XRD). Results of these experiments (FIG. 2) indicate that the lanthanide hydroxide products Nd^{III}(OH)₃ (curve-a), Sm^{III}(OH)₃ (curve-b), Gd^{III}(OH)₃ (curve-c), and Tb^{III}(OH)₃ (curve-d) are crystalline.

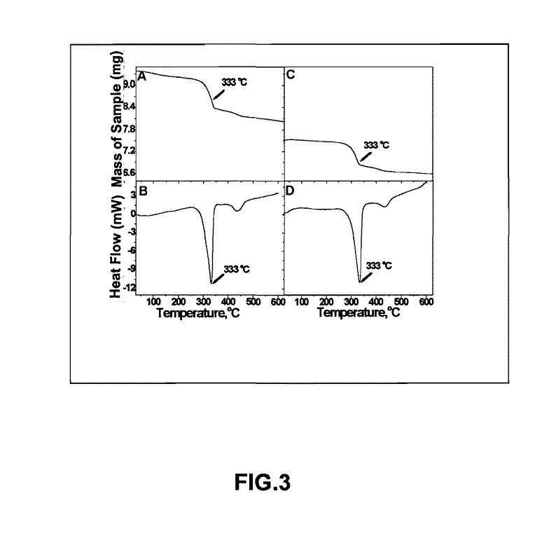

TGA and DSC: To determine the chemical nature (europium hydroxide or europium oxide) of microwave assisted as-synthesized product (60 minutes of microwave irradiation time), TGA and DSC were performed. A representative TGA-DSC profile for as-synthesized product was obtained (FIG. 3A-B). The TGA pattern of as-synthesized product (FIG. 3A) exhibited three distinct weight losses that occur in three steps with an overall weight loss of 16.1% between 30° C. to 600° C. The DSC pattern also exhibited three distinct endothermic peaks at three steps in the same temperature range. The first one, a broad endothermic peak in the temperature range of 30° C. to 200° C. in a DSC curve (FIG. 3A) was associated with the release of 2.4 wt % of residual water, which is physically adsorbed on the surface of the as-synthesized material. The second 8.87 wt % weight loss (compared with a theoretical weight loss of 8.9%) step in the TGA begins around 200° C. and finished at 380° C., and a corresponding well-defined endothermic peak with a sharp peak at 333° C. (FIG. 3B) was observed in the same temperature region. This second weight loss could be ascribed due to the conversion of Eu(OH)₃ to EuO(OH) on dehydration of the hexagonal Eu(OH)₃ (equation-i). The third weight loss of 4.8 wt % (compared with a theoretical weight loss of 4.9%) step in the TGA began around 380° C. and finished at 600° C., and a corresponding well-defined endothermic peak (FIG. 3B) was observed in the same temperature region with a sharp peak at 444° C. This third weight loss could be ascribed due to the decomposition of EuO(OH) to Eu₂O₃ (equation-ii). These second and third steps could be schematically represented as follows:

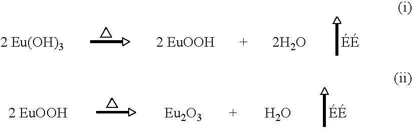

Similar behaviors also were observed for other as-synthesized products, which were obtained after 5 minutes, 10 minutes, 20 minutes, and 40 minutes of microwave heating. Combination of the results of XRD, DSC, and TGA indicated that the as-synthesized materials were europium(III) hydroxide [Eu(OH)₃].

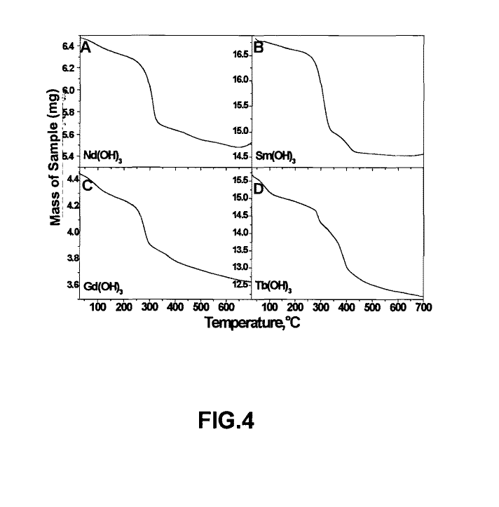

Similarly, thermo gravimetric analysis of other lanthanide hydroxide products (after 60 minutes of microwave irradiation) are presented in FIG. 4A-D. The results indicate that the products are Nd^{III}(OH)₃ (FIG. 4A), Sm^{III}(OH)₃ (FIG. 4AB), Gd^{III}(OH)₃ (FIG. 4C), and Tb^{III}(OH)₃ (FIG. 4D).

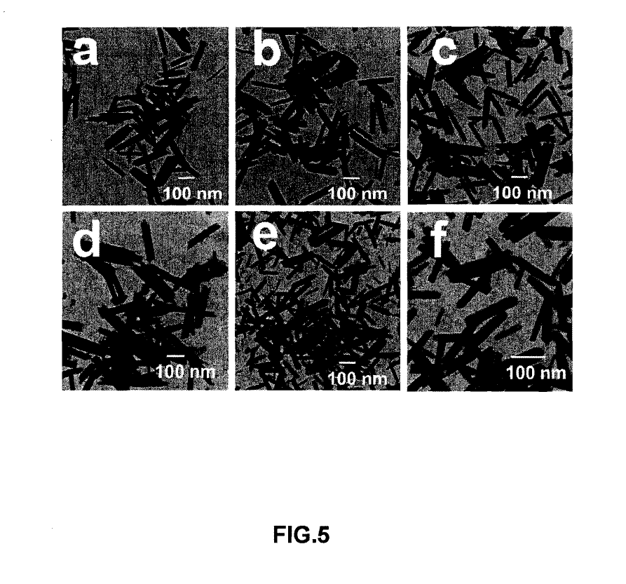

Transmission electron microscopy of nanorods: The morphologies of as-synthesized europium(III) hydroxide [Eu(OH)₃] materials obtained after microwave heating at different times were characterized by TEM (FIG. 5A-F). FIGS. 5A, 5B, 5C, 5D, and 5E contain images of as-synthesize products obtained after 5 minutes, 10 minutes, 20 minutes, 40 minutes, and 60 minutes of microwave heating, respectively. FIG. 5F is a high magnification of FIG. 5E. The TEM images of as-synthesized products revealed that Eu^{III}(OH)₃ material (FIG. 5A-F) entirely consisted of nanorods of diameter from 35 to 50 nm and length from 200 to 300 nm.

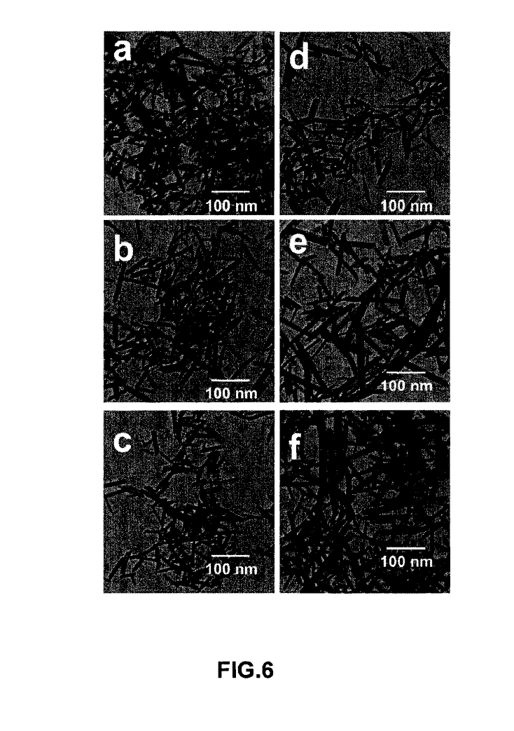

The morphologies of as-synthesized Nd(III) hydroxide [Nd^{III}(OH)₃] materials obtained after microwave heating for different times were characterized by TEM (FIG. 6A-F). FIGS. 6A, 6B, 6C, 6D, 6E, and 6F contain images of as-synthesized products obtained after 1 minute, 5 minutes, 10 minutes, 20 minutes, 40 minutes, and 60 minutes of microwave heating, respectively. The TEM images of as-synthesized products revealed that Nd^{III}(OH)₃ material (FIG. 6A-F) consisted of nanorods with diameters ranging from 35 to 50 nm and lengths ranging from 200 to 300 nm.

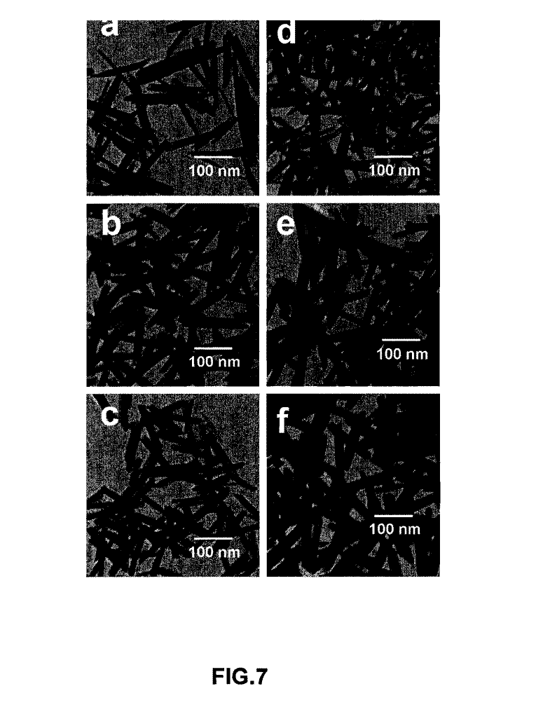

The morphologies of as-synthesized Sm(III) hydroxide [Sm^{III}(OH)₃] materials obtained after microwave heating for different times were characterized by TEM (FIG. 7A-F). FIGS. 7A, 7B, 7C, 7D, 7E, and 7F contain images of as-synthesize products obtained after 1 minute, 5 minutes, 10 minutes, 20 minutes, 40 minutes, and 60 minutes of microwave heating, respectively. The TEM images of as-synthesized products revealed that Sm^{III}(OH)₃ material (FIG. 7A-F) consisted of nanorods with diameters ranging from 35 to 50 nm and lengths ranging from 200 to 300 nm.

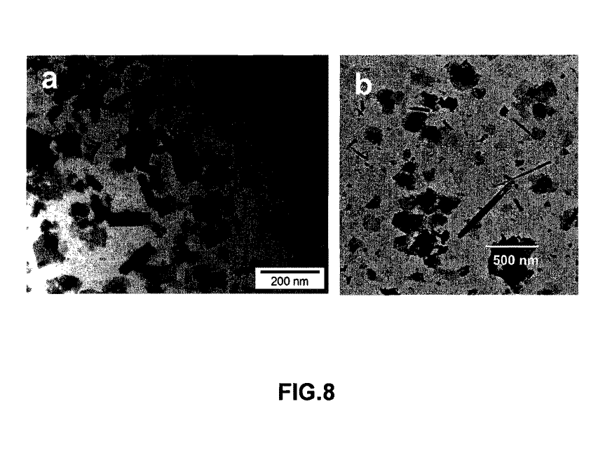

The morphologies of as-synthesized Gd(III) hydroxide [Gd^{III}(OH)₃] and Tb(III) hydroxide [Tb^{III}(OH)₃] materials obtained after 60 minutes of microwave heating were characterized by TEM (FIG. 8A-B). FIGS. 8A and 8B contain images of as-synthesized Gd(III) hydroxide and Tb(III) hydroxide nanomaterials, respectively, obtained after 60 minutes of microwave heating. The TEM images of as-synthesized products revealed that Gd^{III}(OH)₃ (FIG. 8A) and Tb^{III}(OH)₃ material (FIG. 8B) consisted of a mixture of nanoparticles with few nanorods.

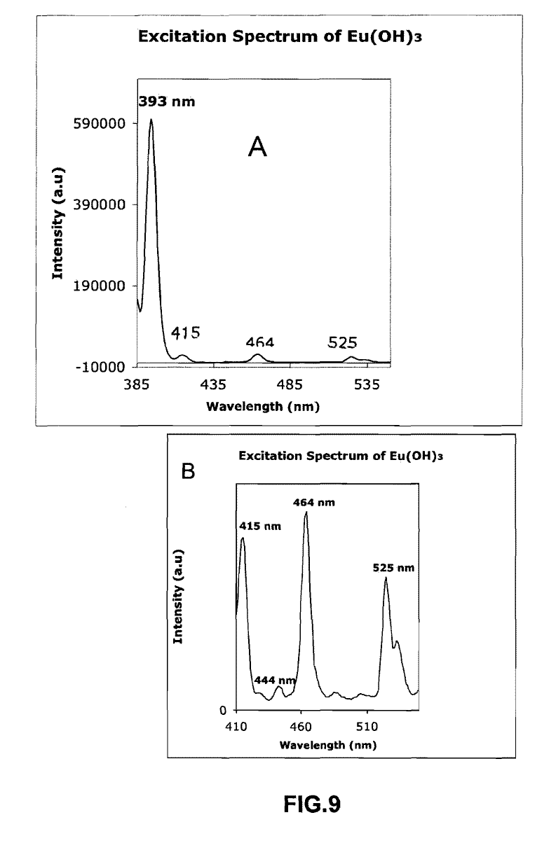

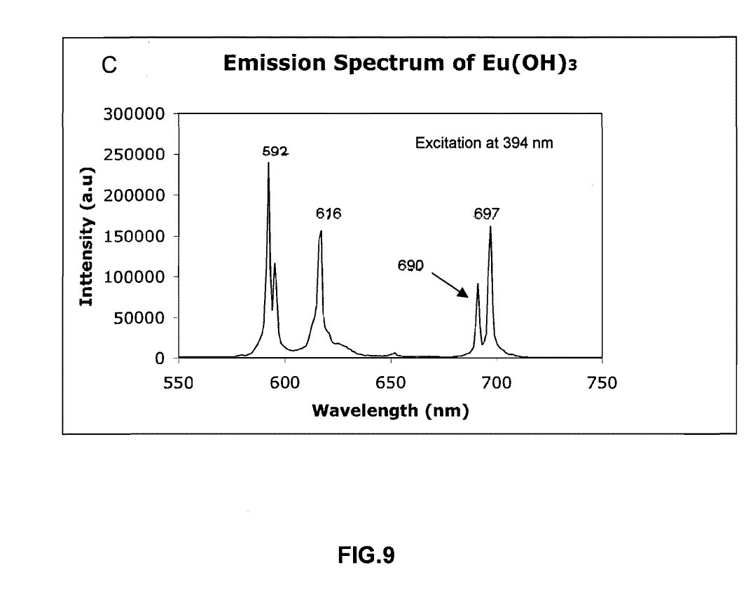

Fluorescence spectroscopy: The excitation and emission spectra of Eu³ ion in Eu^{III}(OH)₃ nanorods arose from transitions of electrons within the 4f shells. The fluorescent emission and excitation spectra of europium hydroxide are shown in FIGS. 9A-B. The excitation spectra were observed at 394 nm (major), 415 nm (minor), 464 nm, and 525 nm (minor) (FIG. 9A) upon the emission wavelength of 616 nm. The main emission spectra for Eu(OH)₃ were observed in 592 nm, 616 nm, 690 nm, and 697 nm (FIG. 9B) after excitation at any of the above wave lengths. The emission spectrum (FIG. 9B) is composed of a ⁵D₀-⁷F_{J} (J=1, 2, 3, 4) manifold of emission lines of Eu³⁺ with the magnetic-dipole allowed ⁵D₀-⁷F₁ transition (588 nm) being the most prominent emission lines.

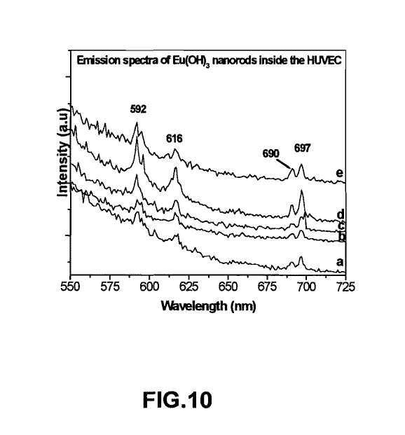

To determine if the fluorescence activity of these Eu^{III}(OH)₃ nanorods remains unchanged even inside the cells, the emission (fluorescence) spectra of the endothelial cells incubated for 24 hours with these nanorods at various concentrations (5-100 μg/mL) were recorded on a Fluorolog-3 Spectrofluorometer after extensive washing with PBS (phosphate buffer saline). Curves a-, b-, c-, d- and e- of FIG. 10 indicate the emission spectra of endothelial cells treated with Eu^{III}(OH)₃ nanorods at the concentrations of 5 μg/mL (curve-a), 10 μg/mL (curve-b), 20 μg/mL (curve-c), 20 μg/mL (curve-d), and 100 μg/mL (curve-e), respectively. Fluorescence emissions were observed in all cases. As these nanorods exhibited their distinct fluorescence properties inside the endothelial cells, it indirectly proved that these nanorods were inside the cells (which was directly proved by TEM).

A number of methods, such as differential interference contrast (DIC) microscopy, confocal microscopy, and transmission electron microscopy (TEM) were used to determine cellular trajectories of nanorods.

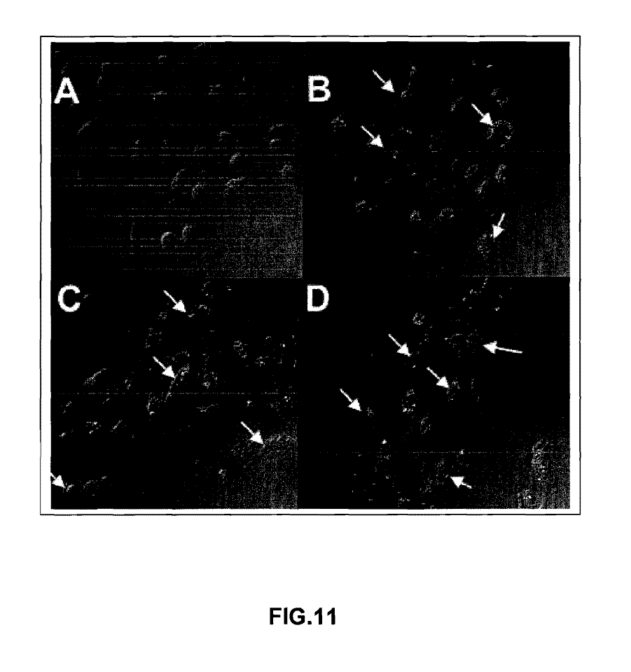

DIC: Differential interference contrast (DIC) microscopy pictures (FIG. 11A-D) revealed a significant difference in contrast between the control cells (FIG. 11A) and the cells treated with Eu^{III}(OH)₃ nanorods at various concentrations (FIGS. 11B-D). These results indirectly proved that Eu^{III}(OH)₃ nanorods can enter the cells.

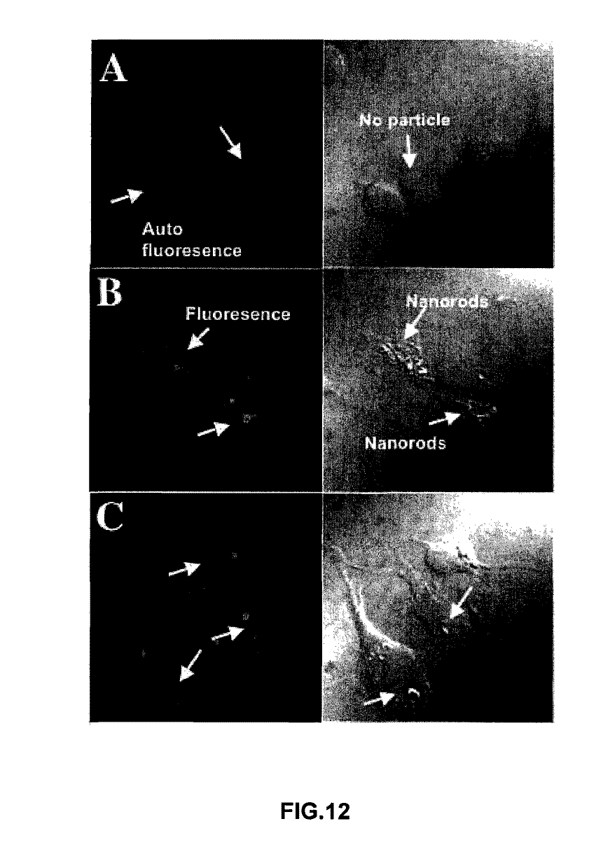

Confocal microscopy: Fluorescence properties of HUVEC loaded with inorganic fluorescent Eu^{III}(OH)₃ nanorods and their corresponding phase images detected by confocal microscopy are presented in the first and second columns of FIG. 12A-C, respectively. The fluorescence (first column) and their corresponding phase images (second column) of the control cells (first row, FIG. 12A) and cells treated with Eu^{III}(OH)₃ nanorods at the concentration of 20 μg/mL (first row, FIG. 12B) and 50 μg/mL (first row, FIG. 12C) are presented in FIG. 12A-C, respectively. The Eu^{III}(OH)₃ nanorods have a useful excitation at the wavelengths of 394 nm, 415 nm, 464 nm, and 525 nm with the maximum intensity at 394 nm. Excitation of Eu^{III}(OH)₃ nanorods at any of the above wavelengths (which are not matching with the laser excitation wavelengths available in the confocal microscope) produce emission peaks at 592 nm, 616 nm, 649 nm, 690 nm and 697 nm, respectively. In this study, confocal fluorescence microscopy images and phase images of cells were collected through the use of a Zeiss LSM 510 confocal laser scan microscope with C-Apochromat 63×/NA 1.2 water-immersion lens, in conjunction with an Argon ion laser (488 nm excitation). The fluorescence emission was collected with a 100× microscope objective, then spectrally filtered using a 515 nm long pass filter. Analysis by confocal laser scanning microscopy (excitation at λ=488 nm) revealed the presence of bright green fluorescence due to presence of Eu³⁺ ions in Eu^{III}(OH)₃ nanorods (FIG. 12B-C) scattered in the cytoplasmic compartments of cells treated with nanorods. Control HUVEC cells (without any nanorods) in FIG. 12A revealed very few green fluorescence inside the cells due to auto fluorescence. Overall, there was a significant difference in fluorescence between control cells and cells treated with these nanorods. These results proved the internalization of Eu^{III}(OH)₃ nanorods inside HUVEC cells. Hence, these nanorods can be used in imaging for the detection and localizations of drugs.

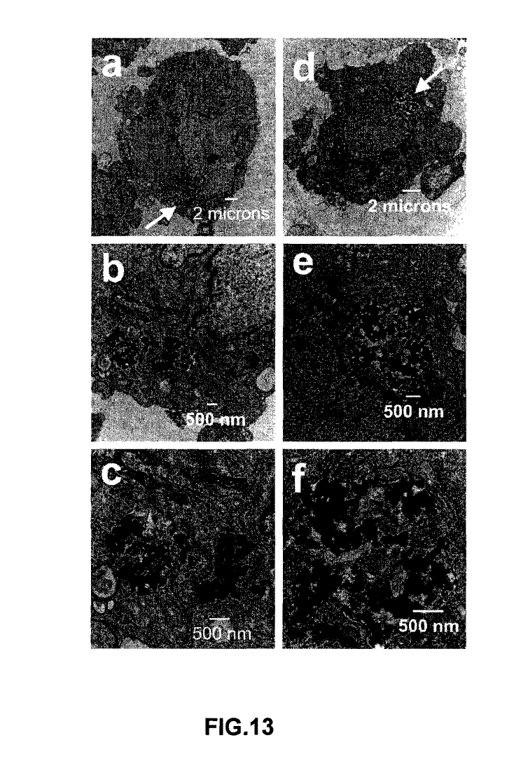

TEM for nanorods inside the cells: The direct proof of internalization of Eu^{III}(OH)₃ nanorods inside the cytoplasmic part of cells was the TEM images of the cells treated with these nanorods at different concentrations. FIG. 13A-C contains TEM images of HUVEC (after cross section) treated with 20 μg/mL of Eu^{III}(OH)₃ nanorods at different magnifications. The images in presented in panels B and C are the higher magnifications of the image presented in panel A. FIGS. 13D-F contain TEM images of HUVEC (after cross section) treated with 50 μg/mL of Eu^{III}(OH)₃ nanorods at different magnifications. The images presented in panels E and F are higher magnifications of the image presented in panel D. These nanorods were visualized inside the cytoplasmic compartments of HUVEC cells. The morphologies of the cells clearly demonstrated that cells were healthy after internalization of these materials (FIG. 13). The cells exhibited a spherical morphology (FIGS. 13A and 13D) because the cells were trypsinized, neutralized with TNS, and fixed in Trumps solution (McDowell and Trump, Arch. Path. Lab. Med., 100: 405 (1976) and Spurr, J. Ultrastruct. Res., 26:31 (1969)) before TEM. FIGS. 13A and 13D also revealed uptake of these nanorods in most of the cells. The incubation of endothelial cells with fluorescent nanorods and subsequent internalization of these nanorods into the cytoplasmic compartment of the cells alter the morphology of the nanorods. This may be because of the very low pH (3.5) of the early and late endosomes.

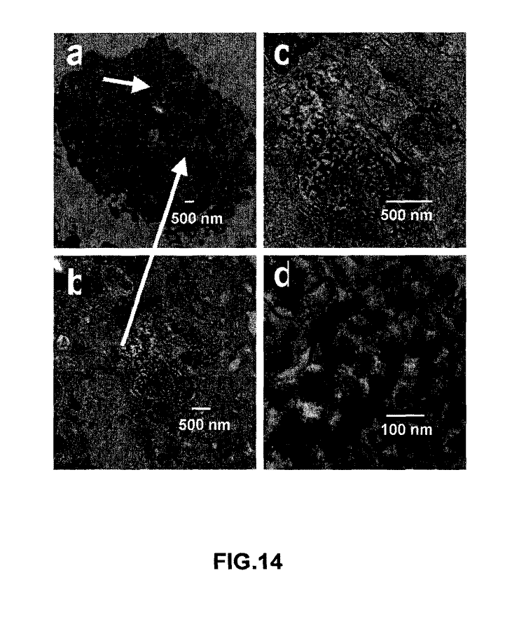

FIGS. 14A-D contain TEM images of HUVEC (after cross section) treated with Tb^{III}(OH)₃ nanorods, at different magnifications. The nanorods were visualized inside the cytoplasmic compartments of HUVEC cells. The morphologies of the cells demonstrated that the cells were healthy after internalization of these materials (FIG. 14). The images presented in panels B, C, and D are the higher magnifications of the image presented in panel A.

Taken together, the results from fluorescence spectroscopy, DIC, confocal microscopy, and TEM indicate that these fluorescent nanorods can be internalized in a cell system and readily visualized by microscopy. These nanorods thus constituted interesting fluorescent probes for the targeting of various molecules to specific cells.

Cell proliferation and viability tests: Before using the inorganic nanorods as a fluorescent label into endothelial cells (HUVEC), the viability of HUVEC was tested after treatment with Eu^{III}(OH)₃ nanorods at different concentrations from 20-100 μg/mL and incubation for 1-2 days to observe apoptosis. There was no difference of cell deaths between the control cells (no treatment) and cells treated with these nanorods as assessed by Trypan Blue exclusion assay. These results indicated that these nanorods were biocompatible with the cells, as they did not affect the cell viability in 24-48 hours.

The Eu^{III}(OH)₃ nanorods' in vitro toxicity was examined in terms of inhibition of proliferation using a [³H] Thymidine incorporation assay (Kang et al., J. Am. Soc. Nephrol., 13:806-816 (2002)) to normal endothelial cells (HUVEC). These nanorods were not toxic to HUVEC (FIG. 15). There were indications that exposure to certain nanomaterials may lead to adverse biological effects that appear to depend upon the material's chemical and physical properties (Gao et al., Curr. Opin. Biotechnol., 16:63 (2005) and Derfus et al., Nano. Lett., 4:11 (2004)). In vivo toxicity can be a factor in determining whether or not fluorescent probes would be approved by regulatory agencies for human clinical use. The results (FIG. 15) from the thymidine incorporation assay using trichloroacetic acid-precipitable radioactivity (Basu et al., Nat. Med., 7:569 (2001)) of HUVEC clearly revealed that these nanorods induced proliferation of the endothelial cells in a dose dependent manner (20-100 μg/mL). Maximum proliferation was observed at the concentrations of 50 μg/mL, and these nanorods were slightly toxic at high concentration (100 μg/mL). When HUVEC cells were treated with a europium^{(III)} nitrate solution (50 μg/mL in TE buffer), no proliferation was observed. In fact, the solution was observed to be slightly toxic to the cells. Experiments were repeated three times. These results demonstrate that Eu^{III}(OH)₃ nanorods are non-toxic to endothelial cells and have some special properties that induce proliferation of the cells.

The Nd^{III}(OH)₃ nanorods also were observed to be non-toxic to HUVEC (FIG. 16). The results (FIG. 16) from the thymidine incorporation assay performed using HUVEC revealed that the nanorods (10-50 μg/mL) do not induce significant proliferation of the endothelial cells in a dose-dependent manner.

The Sm^{III}(OH)₃ nanorods also were observed to be non-toxic to HUVEC (FIG. 17). The results (FIG. 17) from the thymidine incorporation assay performed using HUVEC revealed that the nanorods (20-100 μg/mL) do not induce significant proliferation of the endothelial cells in a dose-dependent manner.

The Tb^{III}(OH)₃ nanorods also were observed to be non-toxic to HUVEC (FIG. 18). The results (FIG. 18) from the thymidine incorporation assay performed using HUVEC revealed that these nanorods do not induce significant proliferation of the endothelial cells in a dose dependent manner (20-100 μg/mL). Tb^{III}(OH)₃ nanorods were compared with terbium nitrate at the concentration of 20 μg/mL.

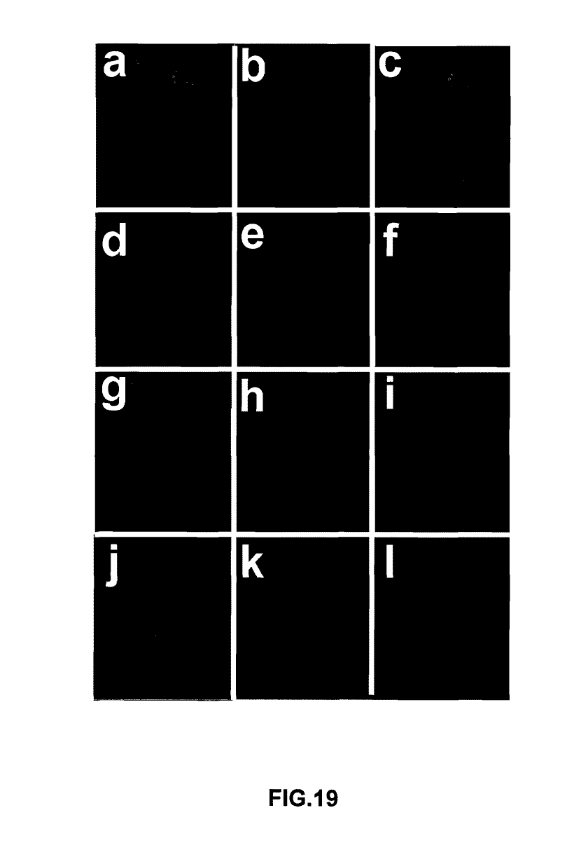

Apoptosis: According to a tunnel based apoptosis assay, the red colored nuclei were tunnel positive (FIG. 19A-C). They were dually stained with DAPI to show the nuclei clearly. As a positive control, cells were treated with 2.5 mM of camptothecin for 4 hours. 100% of red stained nuclei were visible (FIG. 19A). There was no difference (0%) in the number of red stained nuclei between control untreated cells (FIGS. 19D-F) and cells treated with europium hydroxide nanorods at 50 μg/mL (FIGS. 9G-I) or 100 μg/mL (FIGS. 19J-L). About 10% of red stained nuclei were visible in the nanorod-treated (at 100 μg/mL; FIGS. 19J-K) cells compared to control untreated cells. These results demonstrated that there was no induction of apoptosis in HUVEC due to nanorod treatment up to the concentration of 50 μg/mL.

Another group (Kirchner et al., Nano. Lett., 5:331 (2005)) indicated that cellular toxicity of stable nanomaterials is primarily due to aggregation rather than the release of Cd elements. The work provided herein, however, uses nanorods of an entirely different material than cadmium-based materials. Thus, the mode of action of Eu^{III}(OH)₃ nanorods is likely to be different than Cd-based materials, and that is what was observed.

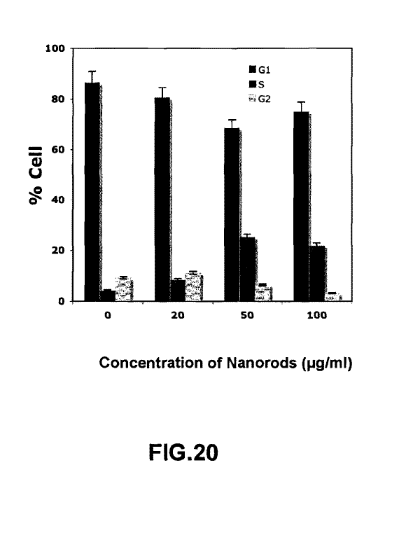

Cell cycle: To investigate the mechanism of HUVEC cell proliferation in the presence of Eu^{III}(OH)₃ nanorods, cell cycle analysis was carried out (FIG. 20). The cell cycle in eukaryotes is broadly classified into four phases, G1-growth and preparation of the chromosomes for replication; S-synthesis of DNA and centrosomes; G2-preparation for mitosis; and M-mitosis, the cell dividing into two daughter cells each with a complete set of chromosomes. Therefore, the proliferation of HUVEC cells should be reflected in cell cycles. Higher populations would be expected in the S-phase, and fewer populations would be expected in the G1-phase (Bhattacharya et al., FASEB J., 19:1692-1694 (2005)). Cell cycle analysis using PI staining in HUVEC cells revealed an increase in the percentage of cells in the S-phase and a significant increase at the concentration of 50 μg/mL of Eu^{III}(OH)₃ nanorods as compared with that of control cells (no treatment; FIG. 20). Conversely, percentage of cells in the S-phase was decreased at the concentration of 100 μg/mL of Eu^{III}(OH)₃ nanorods. These cell cycle results corroborate the results obtained from the proliferation assay.

Map kinase phosphorylation: To further confirm the results obtained from the cell proliferation assay and cell cycle analysis, Western blot analyses of control HUVEC cells (untreated) and HUVEC cells treated with Eu^{III}(OH)₃ nanorods at a concentration of 50 μg/mL for different times (e.g., 5 minutes to 24 hours) were performed. HUVEC cells were treated with vascular endothelial growth factor (VEGF) at the concentration of 10 ng/mL for 5 minutes in positive control experiments (Bhattacharya et al., Nano Lett., 4(12):2479-2481 (2004)).

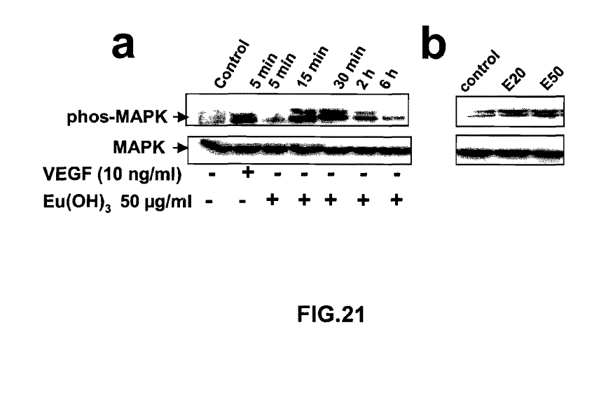

FIGS. 21A and 21B contain data from the Western blot analysis of map kinase phosphorylation in HUVEC cells that were treated with Eu^{III}(OH)₃ nanorods (50 μg/mL) for different lengths of time (panel A), or that were treated with different concentrations of Eu^{III}(OH)₃ nanorods (0, 20, or 50 μg/mL) for 24 hours (panel B). Treatment with Eu^{III}(OH)₃ nanorods upregulated map kinase phosphorylation in a time dependent manner (FIG. 21A). Maximum map kinase phosphorylation occurred at 15 minutes and 30 minutes, and it is more upregulated than VEGF treated samples. After 30 minutes, map kinase phosphorylation decreased with time. Levels came back at 24 hours revealing its biphasic nature.

Conversely, with increasing the concentration of Eu^{III}(OH)₃ nanorods (20-100 μg/mL), map kinase phosphorylation increased, reaching a maximum at 50 μg/mL. Map kinase phosphorylation decreased at 100 μg/mL. These results support the cell proliferation assay results. Therefore, it is concluded that cell proliferation of HUVEC cells after treatment with these nanorods can occur through map kinase phosphorylation pathways.

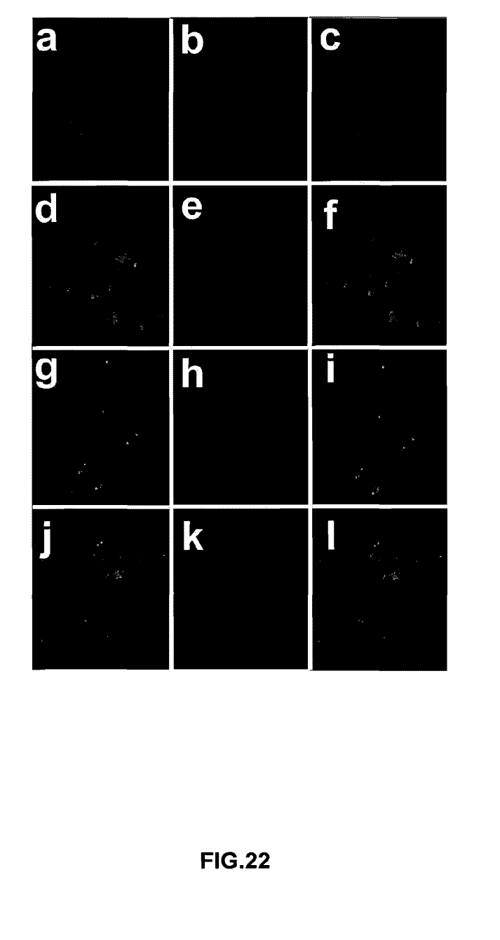

ROS: There was no green fluorescence (FIGS. 22A-C), indicating no ROS formation in the control experiment. The HUVEC cells were induced with 1 μM of tert-butyl hydroperoxide (TBHP) for 1 hour for the positive control experiment of ROS (FIG. 22D-F). The green color fluorescence (FIGS. 22D-F) indicated the formation of oxidation product of carboxy-H₂DCFDA, which indicated reduction of ROS in the cells. The cells were dually stained with Hoechst 33342 to reveal the nuclei clearly as blue. FIGS. 22G-1 and FIGS. 22J-L revealed the generation of ROS in the presence of Eu^{III}(OH)₃ nanorods at the concentration of 20 and 50 μg/mL, respectively. The third column of FIG. 22 (panels C, F, I, and L) revealed merged images of first column (green) and second column (blue). These experiments indicated that the endothelial cells may proliferate through ROS mediated phosphomapkinase pathway.

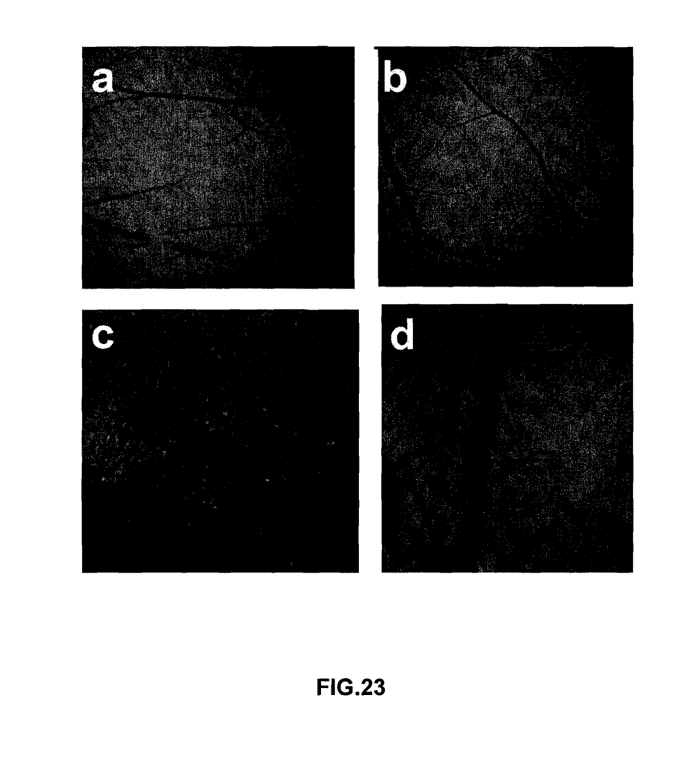

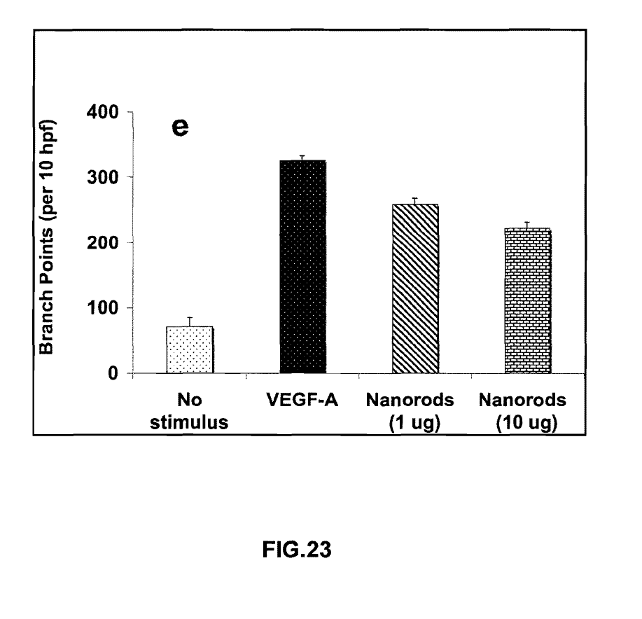

CAM assay (Nanoparticles induce in vivo angiogenesis): To determine the in vivo relevance of the in vitro findings, chick CAM assays were performed to measure nanoparticle-induced angiogenesis. A control experiment where CAMs were treated with only TE (tris-EDTA) buffer solution was performed (FIG. 23A). Eu^{III}(OH)₃ nanorods at 1 μg/mL and 10 μg/mL induced significant angiogenesis (FIGS. 23C-D) when compared to CAMs treated with nanoparticle vehicle. This angiogenic response was about half of that observed with a known pro-angiogenic stimulus of VEGF-A (FIG. 23B). At higher doses of nanoparticles (20 μg/mL), a plaque was found to form on the CAM precluding accurate analysis of vessel branch points. In a number of instances, angiogenesis, remote from this plaque, was noted. These results demonstrate that Eu^{III}(OH)₃ nanorods can exert a significant in vivo angiogenic effect, supporting the in vitro findings. The quantitative data for the angiogenesis assay (CAM assay) using nanorods were also presented as a histogram (FIG. 23E).

In summary, europium(III) hydroxide nanorods, which can be used as inorganic fluorescent materials, were synthesized by a microwave technique, which was simple, fast, clean, efficient, economical, non-toxic, and eco-friendly. The europium(III) hydroxide nanorods retained their fluorescent properties even inside endothelial cells (HUVEC). They were characterized by fluorescence spectroscopy, differential interference contrast microscopy (DIC), confocal microscopy, and transmission electron microscopy (TEM). The nanorods have several advantages over traditional organic dyes as fluorescent labels in biology. For example, these nanorods can promote HUVEC cell proliferation, observed by a [³H]thymidine incorporation assay and cell cycle assay. Further, pro-angiogenic properties of Eu(OH)₃ nanorods were discovered using a CAM assay, which is well established and widely used as a model to examine angiogenesis and anti-angiogenesis.

The europium hydroxide nanorods provided herein can be used as (a) stable and bright fluorescent labels in biology and medicine, (b) pro-angiogenic materials in in vivo systems, and (c) drug delivery vehicles after being conjugated to a drug molecule. In addition, the non-toxic, europium hydroxide nanorods provided herein can be used on heart or limb ischemic tissues for human beings.

### Example 6

### In Vivo Toxicity Studies

Eighteen nude mice (male) were randomized into three groups of 6 animals per group receiving 0 (control group with Tris-EDTA solution injection), 20 (1 mgKg⁻¹ day⁻¹), or 100 μg (5 mgKg⁻¹ day⁻¹) of europium hydroxide [Eu^{III}(OH)₃] nanorods in Tris-EDTA through the IP route of administration for one week. The mice were weighed and examined once per day for any adverse effects or clinical signs throughout the week of regular injections with europium hydroxide nanorods. A mixture of ketamine/xylazine was used to anesthetize mice to facilitate handling. For biochemical and hematological toxicity analysis, blood and serum were collected at the time of sacrifice. Mice in the control groups were sacrificed at the same time as mice of the corresponding experimental group in order to evaluate the effect of the europium hydroxide nanorods in those mice compared to control animals. Mice were sacrificed using the carbon dioxide inhalation method after collection of blood. Hematology analytes included CBC without differential hemoglobin, hematocrit, erythrocytes, mean corpuscular volume (MCV), RBC distribution width, leukocytes and platelet count. Blood chemistry analytes included alkaline phosphates, S (ALP), aspartate aminotransferase (AST), alanine aminotransferase(ALT), creatinine(CR), bilirubin total-S (TBLI), and blood Urea nitrogen (BUN).

In a 7-day toxicity study, intravenous injection of europium hydroxide nanorods (1 mgKg⁻¹ day⁻¹ and 5 mgKg⁻¹ day⁻¹) in Tris-EDTA buffer showed normal hematology (Table 1) and blood chemistry (Table 2). These results indicate that over the above-mentioned dosage, europium hydroxide nanorods appear non-toxic in the in vivo model.

TABLE 1
Blood hematology of mice intravenously injected with 0 (negative control, 0.1 mL
of TE buffer), 1 mgKg⁻¹day⁻¹ (0.1 mL) and 5 mgKg⁻¹day⁻¹ (0.1 mL) of europium
hydroxide nanorods suspended in TE buffer in the blood, sampled at 7 days.

| | Control | 20 μg in | 100 μg in |
|---|---|---|---|
| | (100 μl of | 100 μl | 100 μl |
| Test names | TE buffer) | (5 mgKg⁻¹day⁻¹) | (5 mgKg⁻¹day⁻¹) |
| CBC without differential | 9.5 ± 3.8 | 11.9 ± 1.3 | 11.5 ± 1.2 |
| hemoglobin (g/dL) | | | |
| Hematocrit (%) | 32 ± 4.9 | 35.3 ± 3.8 | 34.9 ± 3.1 |
| Erythrocytes [×10(12)/L] | 6.3 ± 0.9 | 7.1 ± 0.6 | 6.7 ± 0.8 |
| MCV (fL) | 50.5 ± 3.6 | 49.6 ± 2.6 | 51.9 ± 1.7 |
| RBC Distrib Width (%) | 15.5 ± 1.2 | 16.2 ± 0.8 | 15.1 ± 0.4 |
| Leukocytes [×10(9)/L] | 1.3 ± 0.9 | 1.0 ± 0.4 | 0.7 ± 0.2 |
| Platelet count [×10(9)/L] | 599.6 ± 389.3 | 480.7 ± 317.4 | 476.0 ± 376.1 |

Six animals were used per measurements, and all values were within the normal range.

TABLE 2
Serum clinical chemistry of mice intravenously injected with 0
(negative control, 0.1 mL of TE buffer), 1 mgKg⁻¹day⁻¹ (0.1 mL) and 5 mgKg⁻¹
day⁻¹ (0.1 mL) of europium hydroxide nanorods suspended in
TE buffer in the blood, sampled at 7 days.

| | Control | 20 μ in | 100 μg in |
|---|---|---|---|
| | (100 μl of | 100 μl | 100 μl |
| Test names | TE buffer) | (5 mgKg⁻¹day⁻¹) | (5 mgKg⁻¹day⁻¹) |
| Alkaline phosphates, S (U/L) | 143.2 ± 13.6 | 99.3 ± 13.7 | 91.0 ± 4.1 |
| Aspartate Aminotransferase (U/L) | 140.5 ± 41.6 | 184.0 ± 96.1 | 209.7 ± 33.5 |
| Alanine Aminotransferase (U/L) | 32.5 ± 6.6 | 47.3 ± 12.7 | 39.0 ± 5.7 |
| Creatinine, P/S (mg/dL) | 0.1 ± 0.0 | 0.1 ± 0.0 | 0.1 ± 0.0 |
| Bilirubin total, S (mg/dL) | 0.2 ± 0.0 | 0.1 ± 0.0 | 0.2 ± 0.0 |
| Bld Urea nitrogen (BUN) | 22.8 ± 2.4 | 22.3 ± 0.5 | 24.3 ± 2.4 |

Six animals were used per measurements, and all values were within the normal rage.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for making lanthanide hydroxide nanoparticles, wherein said method comprises microwave heating a mixture of lanthanide nitrate and aqueous ammonium hydroxide.

2. The method of claim 1, wherein said lanthanide is europium.

3. The method of claim 1, wherein said nanoparticles are europium hydroxide nanorods.

4. The method of claim 3, wherein said europium hydroxide nanorods are between 10 and 500 nm in length.

5. The method of claim 3, wherein the diameter of said europium hydroxide nanorods are between 1 and 100 nm.

6. Lanthanide hydroxide nanoparticles having a length between 10 and 500 nm and a diameter between 1 and 100 nm, wherein said nanorods promote angiogenesis.

7. The lanthanide hydroxide nanoparticles of claim 6, wherein said lanthanide is europium.

8. The lanthanide hydroxide nanoparticles of claim 6, wherein said nanoparticles are europium hydroxide nanorods.

9. A method of promoting angiogenesis, wherein said method comprises contacting cells with lanthanide hydroxide nanoparticles.

10. The method of claim 9, wherein said lanthanide is europium.

11. The method of claim 9, wherein said nanoparticles are europium hydroxide nanorods.

12. The method of claim 11, wherein said europium hydroxide nanorods are between 10 and 500 nm in length.

13. The method of claim 11, wherein the diameter of said europium hydroxide nanorods are between 1 and 100 nm.
